SEQUENCE LISTING (4 sequences)

SEQ ID NO: 1 - DNA, 11 bp, Artificial Sequence
  P oligo
agtcggagtct

SEQ ID NO: 2 - DNA, 11 bp, Artificial Sequence
  T oligo
agactccgact

SEQ ID NO: 3 - RNA, 11 nt, Artificial Sequence
  T oligo RNA form
agacuccgacu

SEQ ID NO: 4 - RNA, 17 nt, Artificial Sequence
  T-3U oligo
agcgcuagacuccgacu

(19) United States
(12) Patent Application Publication
(10) Pub. No.: US 2022/0228139 A1
(43) Pub. Date: Jul. 21, 2022

(54) METHOD FOR AMPLIFYING AND DETECTING RIBONUCLEIC ACID (RNA) FRAGMENTS

(71) Applicant: ACADEMIA SINICA, Taipei City (TW)

(72) Inventors: Kuo-Ping CHIU, Taipei City (TW); Hsin-Chieh SHIAU, Taipei (TW); Zee Hong GOH, Taipei City (TW)

(73) Assignee: ACADEMIA SINICA, Taipei City (TW)

(21) Appl. No.: 17/612,635

(22) PCT Filed: May 21, 2020
(86) PCT No.: PCT/US2020/033929

§ 371 Date: Nov. 19, 2021

Related U.S. Application Data
(60) Provisional application No. 62/850,651, filed on May 21, 2019.

(51) Int. Cl. C12N 15/10; C12Q 1/6855; C12Q 1/686; C12N 9/22

(52) U.S. Cl. CPC C12N 15/1096; C12N 9/22; C12Q 1/686; C12Q 1/6855

(57) ABSTRACT

The present invention relates to a method for amplifying and detecting ribonucleic acid (RNA) fragments. In particular, the method of the present invention comprises conversion of RNA fragments to cDNA and DNA amplification. The present invention also provides a kit for performing the method as described herein.

### RELATED APPLICATIONS

This application claims the benefit of U.S. provisional application No. 62/850,651, filed May 21, 2019 under 35 U.S.C. § 119, the entire content of which is incorporated herein by reference.

### TECHNOLOGY FIELD

The present invention relates to a method for amplifying and detecting ribonucleic acid (RNA) fragments. In particular, the method of the present invention comprises conversion of RNA fragments to cDNA and DNA amplification. The present invention also provides a kit for performing the method as described herein.

### BACKGROUND

RNAs are important genetic material involved in gene expression and regulation. In particular, cell-free RNAs (cfRNAs) in biofluids (e.g., blood, saliva, urine, etc.) carry important genetic information with biological and medical relevance and are thus becoming valuable noninvasive specimens for diagnosis of many diseases. However, cfRNAs are very diverse, with structures and functions remain largely unknown. In addition, since cfRNAs are normally present in biofluids in low quantity and may degrade or become fragmented easily, it has been a challenge to detect or analyze cfRNAs using current methods.

Some conventional technologies have been developed for RNA detection, validation and quantification. In general, RNAs are isolated from biological samples and converted into complementary DNAs (cDNAs) by reveres transcription (RT), followed by amplification using conventional or quantitative polymerase chain reaction (qPCR). Conventional PCR methods for DNA amplification requires two or more paired oligonucleotide primers, each pair comprising a forward primer and a reverse primer to specifically define the boundaries of a particular target nucleic acid sequence to be amplified. For example, New England Biolabs (NEB) commercializes a method with kit (NEBNext small RNA library preparation kit) which generates cDNA fragments with different adapters at 5′-end and 3′-end for two different primers to bind (see step f in FIG. 1), which however may cause efficiency problem. In this connection, Ferrero et al. described small non-coding RNA profiling in human biofluids and surrogated tissues from healthy individuals (Ferrero et al., 2018). Yuan et al. described plasma extracellular RNA profiles in healthy and cancer patients (Yuan et al., 2016). Everaert et al. described performance assessment of total RNA sequencing of human biofluids and extracellular vesicles (EVs) (Everaert et al., 2019). These methods have limitations resulted from various aspects of the cfRNAs to be assessed, including low quantity, short fragment length, large variety or quick degradation. As such, a comprehensive method for thorough assessment of all RNA species in a sample is highly desired.

### SUMMARY

The present invention provides a new method for RNA assessment.

In general, the present invention provides an improved PCR-based technique for assessment of RNAs which features reverse transcription of RNAs to generate cDNA products having a single-type (homogenous) adaptor at both termini to permit DNA amplification with a single primer as both forward and reverse primers. The method of the present invention needs less RNA amount as initial input and is particularly useful for detecting trace amount of RNA molecules, and thus subsequent detection with target specific probes can be carried out with increased sensitivity. Furthermore, the method of the present invention achieves a comprehensive RNA profiling for total RNAs covering a large variety of RNA species without bias where the amplified cDNAs maintain the relative quantity of the corresponding RNA fragments in the original sample, which at least provides the advantages that subsequent detection with target specific probes can be carried out with increased sensitivity and less false negatives.

Specifically, the present invention provides a method of converting a linear, single-stranded RNA (ssRNA) fragment to a DNA fragment and amplifying the DNA fragment. The said method comprises the following steps:

(a) removing 5′ phosphate from the ssRNA fragment to produce a de-phosphorylated ssRNA fragment;

(b) ligating a P oligo (DNA), a single-stranded DNA having a P oligo sequence and carrying a 5′-phosphate, to 3′-end of the de-phosphorylated ssRNA fragment to form a ssRNA-P oligo (DNA) strand;

(c) performing a first reverse transcription by using the 5′-ssRNA-P oligo (DNA)-3′ strand as a template and adding a T oligo (DNA), a single-stranded DNA having a T oligo sequence that is complementary and anneals to the P oligo (DNA), as a primer, to synthesize a complementary DNA (cDNA) strand that is complementary to the ssRNA fragment to produce a 5′-T oligo (DNA)-cDNA-3′ strand and thus form an initial RNA/DNA hybrid composed of said ssRNA-P oligo (DNA) strand and the cDNA-T oligo (DNA) strand;

(d) ligating a T oligo (RNA), a single-stranded RNA complementary to the P oligo (DNA), to 5′-end of the 5′-ssRNA-P oligo (DNA)-3′ strand in the initial RNA/DNA hybrid, to form a 5′-T oligo (RNA)-ssRNA-P oligo (DNA)-3′ strand and thus form an intermediate RNA/DNA hybrid composed of said 5′-T oligo (RNA)-ssRNA-P oligo (DNA)-3′ strand and the 5′-T oligo (DNA)-cDNA-3′ strand, having a non-complementary T oligo (RNA) overhang;

(e) performing a second reverse transcription using the non-complementary T oligo (RNA) overhang as an extended template to obtain a complete cDNA strand having the T oligo sequence at the 5′-end and the P oligo sequence at the 3′-end and thus form a complete RNA/DNA hybrid of said 5′-T oligo (RNA)-ssRNA-P oligo (DNA)-3′ strand and said complete cDNA strand;

(f) removing the T oligo (RNA) and the ssRNA fragment from the RNA/DNA hybrid to produce a partial, double-stranded DNA comprising said complete cDNA strand partially hybridized at its 5′-end with the P oligo (DNA); and

(g) performing a T oligo-primed polymerase chain reaction (TOP-PCR) using such extended cDNA strand as a PCR template and a T oligo primer having the T oligo sequence to prime synthesis of a double-stranded cDNA product.

In some embodiments, the ssRNA fragment comprises a nucleic acid sequence indicative of a healthy/diseased state of a subject.

In some embodiments, the ssRNA fragment is present in a sample from a subject, e.g., a diseased subject.

In some embodiments, the sample is obtained from a body fluid sample, including, but not limited to, a sample from blood, urine, saliva, tears, sweat, breast milk, nasal secretions, amniotic fluid, semen, or vaginal fluid of the subject.

In some embodiments, the ssRNA fragment is cell-free RNAs (cfRNAs). In particular, the cfRNAs are RNAs in vesicles (vc-RNAs) such as those in exosomes, microvesicles, or endosomes.

In some embodiments, prior to step (d), the ssRNA-P oligo (DNA) strand is phosphorylated.

In some embodiments, in step (g), the T oligo primer is the only primer used in the PCR reaction.

In some embodiments, the ssRNA fragment is present as an initial input (total RNA) in an amount of 0.01 ng to 100 ng or less (e.g. 0.01 ng to 10 ng or less).

In some embodiments, the ssRNA fragment is present as an initial input (total RNA) in an amount of about 90 ng, 80 ng, 70 ng, 60 ng, 50 ng, 40 ng, 30 ng, 20 ng, 10 ng, 5 ng, 2.5 ng, 1 ng or less.

In some embodiments, the ssRNA fragment is present as an initial input (total RNA) in an amount of 0.01 ng to 100 ng or more (e.g. 0.1 ng to 100 ng or more, 10 ng to 100 ng or more, or 1 microgram or more).

In some embodiments, the method of the present invention further comprises detecting the amplified cDNA product by diagnostic or clinical devices (e.g., mass spectrometry, hybridization or sequencing).

In some embodiments, the method of the present invention may include one or more purification steps.

In some embodiments, the method of the present invention does not include a purification step.

The present invention also provides a method for RNA assessment, comprising

(i) providing a biofluid sample from a subject, wherein the biofluid includes ssRNA fragments;

(ii) performing the RNA TOP-PCR method of the present invention as described herein to convert the ssRNA fragments to corresponding DNA fragments and amplify such DNA fragments; and

(iii) analyzing the amplified DNA fragments for measurement of one or more characteristics of the amplified DNA fragments.

In some embodiments, the (iii) analyzing step includes sequencing, mapping and/or alignment.

The present invention also provides a kit for performing the RT-PCR method as described herein, comprising

(i) a de-phosphorylation reagent comprising an alkaline phosphatase and a de-phosphorylation buffer;

(ii) a ligation reagent comprising a ligase, a ligation buffer, the P oligo (DNA) and the T oligo (RNA);

(iii) a phosphorylation reagent comprising a kinase and a kinase buffer;

(iv) a reverse transcription reagent comprising a reverse transcriptase (RT), an RT buffer, dNTP and the T oligo (DNA);

(v) a RNA digestion reagent comprising an RNase and an RNase buffer; and

(vi) a PCR reagent comprising a DNA polymerase, a PCR buffer, dNTP, and the T oligo primer.

The details of one or more embodiments of the invention are set forth in the description below. Other features or advantages of the present invention will be apparent from the following detailed description of several embodiments, and also from the appending claims.

### BRIEF DESCRIPTION OF THE DRAWINGS

The foregoing summary, as well as the following detailed description of the invention, will be better understood when read in conjunction with the appended drawings. For the purpose of illustrating the invention, certain embodiments are shown in the drawings which are presently preferred. It should be understood, however, that the invention is not limited to the precise arrangements and instrumentalities shown.

FIG. 1 shows the comparison of the method of the present invention, RNA T oligo-primed polymerase chain reaction (RNA TOP-PCR), to the NEB method. The first two steps (A-B and a-b) are similar except that the RNA TOP-PCR method of the present invention starts with much less amount of total RNA. Then, two experimental procedures divert substantially: For the RNA TOP-PCR method of the present invention, first strand cDNA synthesis (C) is followed by ligation of T oligo (in RNA form) to the 5′ end of the RNA strand (D) and then reverse transcription to complete the full-length of first strand cDNA (E). Then, RNA portion is digested (F) before TOP-PCR amplification (G). For NEB's method, 3′ primer hybridization (c) is followed by 5′ single-stranded RNA (ssRNA) adapter ligation (d), synthesis of full-length first strand cDNA (e), which is then subjected to PCR amplification (f) using their conditions together with two different PCR primers. Moreover, their PCR products need to be size-selected to remove adapter dimers, while the TOP-PCR method does not require size selection.

FIG. 2 shows the workflow of EV-RNA assessment in certain embodiments of the present invention.

### DETAILED DESCRIPTION

Unless defined otherwise, all technical and scientific terms used herein have the same meanings as is commonly understood by one of skill in the art to which this invention belongs.

As used herein, the articles “a” and “an” refer to one or more than one (i.e., at least one) of the grammatical object of the article. By way of example, “an element” means one element or more than one element.

The term “comprise” or “comprising” is generally used in the sense of include/including which means permitting the presence of one or more features, ingredients or components. The term “comprise” or “comprising” encompasses the term “consists” or “consisting of.”

As used herein, “around”, “about” or “approximately” can generally mean within 20 percent, particularly within 10 percent, and more particularly within 5 percent of a given value or range. Numerical quantities given herein are approximate, meaning that the term “around”, “about” or “approximately” can be inferred if not expressly indicated.

The term “polynucleotide” or “nucleic acid” refers to a polymer composed of nucleotide units. Polynucleotides include naturally occurring nucleic acids, such as deoxyribonucleic acid (“DNA”) and ribonucleic acid (“RNA”) as well as nucleic acid analogs including those which have non-naturally occurring nucleotides. Polynucleotides can be synthesized, for example, using an automated DNA synthesizer. The term “nucleic acid” typically refers to large polynucleotides. Polynucleotides or nucleic acids can be either single-stranded (e.g. ssRNA or a single-stranded cDNA) or double-stranded (e.g. a RNA/DNA duplex or dsDNA). It will be understood that when a nucleotide sequence is represented by a DNA sequence (i.e., A, T, G, C), this also includes an RNA sequence (i.e., A, U, G, C) in which “U” replaces “T.” The term “oligonucleotide” refers to a relatively short nucleic acid fragment, typically less than or equal to 150 nucleotides long e.g., between 5 and 150. Oligonucleotides can be designed and synthesized as needed. In the case of a primer, it is typically between 5 and 50 nucleotides, particularly between 8 and 30 nucleotides in length. In the case of a probe, it is typically between 10 and 100 nucleotides, particularly between 30 and 100 nucleotides in length. The term “P oligo” as used herein can refer to an oligonucleotide carrying a 5′-phosphate for ligating to 3′-end of RNA fragments. The term “T oligo” as used herein can refer to an oligonucleotide complementary to P-oligo.

As used herein, the term “complementary” refers to the topological compatibility or matching together of interacting surfaces of two polynucleotides. Thus, the two molecules can be described as complementary, and furthermore the contact surface characteristics are complementary to each other. A first polynucleotide is complementary to a second polynucleotide if the nucleotide is sequence of the first polynucleotide is identical to the nucleotide sequence of the polynucleotide binding partner of the second polynucleotide. Thus, the polynucleotide whose sequence 5′-TATAC-3′ is complementary to a polynucleotide whose sequence is 5′-GTATA-3′.”

As used herein, target nucleic acids refer to particular nucleic acids of interest being detected in a sample. Specifically, the target nucleic acids include RNA, particularly cfRNA, including mRNA, tRNA, rRNA, miRNA, cfRNA, and/or vcRNA. Target nucleic acids may derive from any sources including naturally occurring sources or synthetic sources. For example, target nucleic acids may be from animal or pathogen sources including, without limitation, mammals such as humans, and pathogens such as bacteria, viruses and fungi. Target nucleic acids can be obtained from any body fluids or tissues (e.g., blood, urine, skin, hair, stool, and mucus), or an environmental sample (e.g., a water sample or a food sample). In some embodiments, target nucleic acids can be a collection of nucleic acid molecules of the same origin (e.g., from the same gene of normal or diseased subject or pathogens) but in various length.

As used herein, the term “cell free RNA(s)” or cfRNA(s) refers to any types of RNAs that are circulating in the bodily fluid of an individual, but are not present inside of cell body or a nucleus. The cell free RNAs have emerged as valuable invasive biomarkers for early detection, prognosis or monitoring of diseases, particularly cancers. RNAs are unstable that are sensitive to degradation by ribonucleases. Cell-free RNAs circulating in the bodily fluid have been found to be encapsulated within extracellular vesicles (EVs) or to exist in a vesicle-free form associated with lipoproteins or other RNA binding proteins. The cell free RNAs can be any type of RNA, including but are not limited to messenger RNA (mRNA), transfer RNA (tRNA), ribosomal RNA (rRNA) and non-coding RNA (including long non-coding RNA (IncRNA) exceeding 200 nucleotides and small non-coding RNA (SncRNA) smaller than 200 nucleotides). Examples of SncRNA include small interfering RNA (siRNA), microRNA (miRNA), Vault RNAs (vtRNA) and Y-RNA etc. Cell free RNAs can be those in full length or fragmented, for example, a fragment of mRNA (e.g., at least 80% of full-length, at least 70% of full length, at least 60% of full length, at least 50% of full length, at least 40% of full length etc.) encoding one or more proteins (e.g. cancer-related proteins, inflammation-related proteins, signal transduction related proteins, energy metabolism related proteins). The RNA(s) may vary broadly in size, for example, ranging from about 10 bases or less to about 3,000 bases or more, specifically including the populations of 70-80 bases, 80-90 bases, 90-110, bases, and 150-170 bases, for example.

Suitable methods are available to isolate cell free RNA. Typically, cell free RNA is isolated from a biofluid e.g. whole blood preferably processed as plasma or serum, or any other fluids e.g. saliva, ascites fluid, urine, spinal fluid, etc., which are deemed appropriate as long as cell free RNA is present in such fluids. In some typical embodiments, whole blood is centrifuged to fractionate plasma. The plasma thus obtained is then separated and centrifuged to remove cell debris. Cell free RNA is extracted from the plasma using commercialized reagents (e.g. Qiagen reagents). The resultant RNA samples can be frozen prior to further processing.

As used herein, the term “trace” or “low” amount with respect to nucleic acids in a sample may refer to an amount relatively less than that as used in a conventional method for assessment of the nucleic acids. For example, a trace amount relevant to RNAs to be analyzed in a biological sample may refer to about 0.01 ng to 100 ng or less (e.g. 0.01 ng to 10 ng or less, or a few RNA molecules or even one single RNA molecule).

As used herein, the term “primer” refers to oligonucleotides that can be used in an amplification method, such as a polymerase chain reaction (PCR), to amplify a target nucleotide sequence. In a conventional PCR, at least one pair of primers including one forward primer and one reverse primer are required to carry out the amplification. Typically, for a target DNA sequence consisting of a (+) strand and a (−) strand to be amplified, a forward primer is an oligonucleotide that can hybridize to the 3′ end of the (−) strand and can thus initiate the polymerization of a new (+) strand under the reaction condition; whereas a reverse primer is an oligonucleotide that can hybridize to the 3′ end of the (+) strand under the reaction condition and can thus initiate the polymerization of a new (−) strand under the reaction condition. Specifically, as an example, a forward primer may have the same sequence as the 5′ end of the (+) strand, and a reverse primer may have the same sequence as the 5′ end of the (−) strand. Normally, a forward primer and a reverse primer used for amplification of a target nucleic acid sequence are different from each other in sequence. As used herein, a “single” primer refers to only one type of primer, all of which have the same sequence, instead of a pair of primers having distinct sequences, one being a forward primer and the other being a reverse primer.

The term “hybridization” as used herein shall include any process by which a strand of nucleic acid joins with a complementary strand through base pairing. Relevant methods are well known in the art and described in, for example, Sambrook et al., Molecular Cloning: A Laboratory Manual, 2^{nd} ed., Cold Spring Harbor Laboratory Press (1989), and Frederick M. A. et al., Current Protocols in Molecular Biology, John Wiley & Sons, Inc. (2001). Typically, stringent conditions are selected to be about 5 to 30° C. lower than the thermal melting point (Tₘ) for the specified sequence at a defined ionic strength and pH. More typically, stringent conditions are selected to be about 5 to 15° C. lower than the Tₘ for the specified sequence at a defined ionic strength and pH. For example, stringent hybridization conditions will be those in which the salt concentration is less than about 1.0 M sodium (or other salts) ion, typically about 0.01 to about 1 M sodium ion concentration at about pH 7.0 to about pH 8.3 and the temperature is at least about 25° C. for short probes (e.g., 10 to 50 nucleotides) and at least about 55° C. for long probes (e.g., greater than 50 nucleotides). An exemplary non-stringent or low stringency condition for a long probe (e.g., greater than 50 nucleotides) would comprise a buffer of 20 mM Tris, pH 8.5, 50 mM KCl, and 2 mM MgCl₂, and a reaction temperature of 25° C.

The term “reverse transcription” as used herein mean generation of complementary DNA (cDNA) from a RNA template, which is usually performed by an enzyme such as the reverse transcriptase and requires a primer be annealed to the RNA template.

A “single,” “homogenous” or “universal” primer means only one type of primer with the same sequence is present, instead of a pair of primers, in the PCR reaction. The term “heterogeneous primers” means at least one paired primers each member having different sequences from each other are present in the PCR reaction.

As used herein, the term “adaptor” refers to an oligonucleotide that can be ligated to the ends of a nucleic acid molecule. An adaptor may be 10 to 50 bases in length, preferably 10 to 30 based in length, more preferably 10 to 20 based in length. Lower than 10 nucleotide in length may decrease specificity for annealing. Higher than 20 nucleotides in length may not be cost-effective. The term a “homogeneous” adaptor means one single type of adaptor for ligating to both ends of a double stranded nucleic acid molecule. The term a “heterogeneous” adaptor means at least two types of adaptors that have different nucleotide sequences from each other, one present at 5′ end and the other present at 3′end of a double stranded nucleic acid molecule. In the present invention, a homogenous adaptor formed by a P oligo and a T oligo is used. In one embodiment of the invention, the T oligo has the sequence: 5′-AGACTCCGACT-3′ (SEQ ID NO: 2); and the P oligo has the corresponding sequence: 5′-AGTCGGAGTCT-3′ (SEQ ID NO: 1). The sequence can be in RNA form (which base U may be used instead of base T in some positions).

The present invention provides an improved technology for RNA conversion and cDNA amplification called “RNA T oligo-primed polymerase chain reaction (RNA TOP-PCR)” which is particularly useful for comprehensive unbiased amplification of trace amount of linear, single-stranded RNA. Compared to a conventional RT-PCR technology, which generates cDNA fragments with different adaptors at the 5′-end and the 3′-end and thus the subsequent amplification requires two different primers, the method of the present invention generates cDNA fragments with a homogenous (single type) adaptor made of a P oligo and a T oligo complementary to each other, and then the resultant cDNA fragments can be amplified with a single T oligo primer annealing to the P oligo of the homogenous adaptor. By doing so, the initial input of RNA fragments can be lower, the efficiency of the RNA to DNA conversion and DNA amplification is increased. In addition, all the RNA fragments in the sample can be equally amplified and subsequent detection with target specific probes can be carried out with increased sensitivity. According to the method of the present invention, a trace amount of RNA samples is sufficient, for example, about 0.01 ng to 100 ng or less (e.g. 90 ng or less, 80 ng or less, 70 ng or less, 60 ng or less, 50 ng or less, 40 ng or less, 30 ng or less, 20 ng or less, 10 ng or less, 5 ng or less, 1 ng or less, 0.5 ng or less, 0.1 ng or less, 0.01 ng or less, or a few RNA molecules or even a single RNA molecule) as initial input in a sample to be detected. It is understandable that the method of the present invention is also applicable for a higher amount of RNA samples, for example, 0.01 ng to 100 ng or more (e.g. 0.1 ng to 100 mg or more, 10 ng to 100 ng or more, or 1 microgram or more).

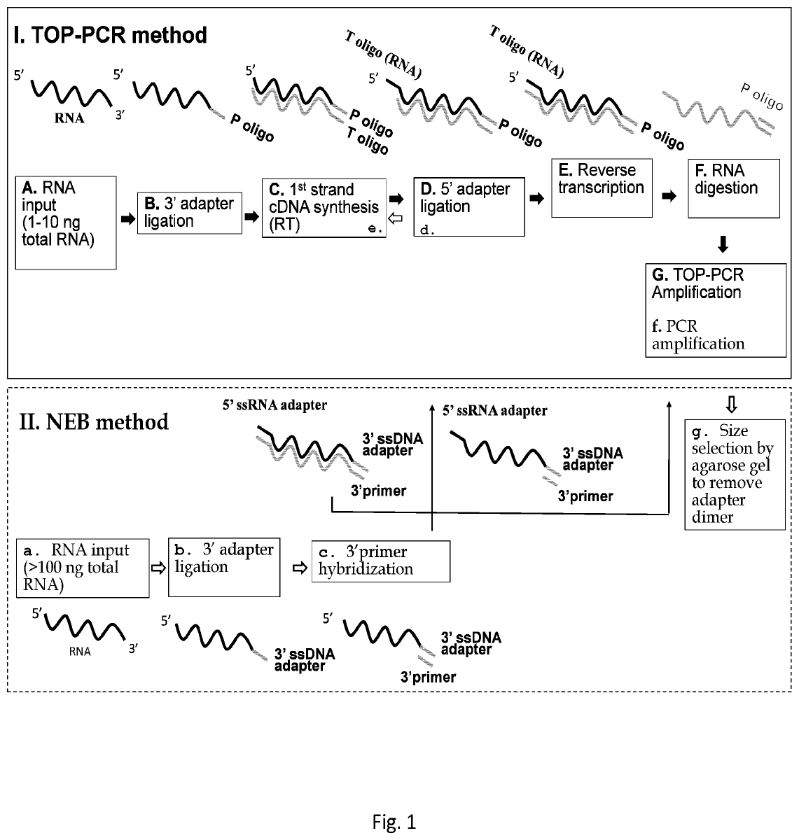

FIG. 1 is a diagram showing the procedures of the method of the present invention (steps A to G). Step A performs 5′ dephosphorylation of cfRNA. Step B performs 3′ligation of cfRNA to P oligo. Step C performs the first cDNA synthesis by reverse transcription. Step D performs 5′ adaptor ligation of cfRNA with T oligo (RNA form). Step E performs extended reverse transcription. Step F performs RNA digestion. Step G performs the TOP-PCR amplification. The TOP-PCR technology has been described in, for example, U.S. Patent Application Publication No. 20160298172 (i.e. U.S. Pat. No. 10,407,720), the entire content of which is incorporated herein by reference. Details are described below in the examples.

The RNA TOP-PCR of the present invention is particularly designed for amplification of low abundance RNA fragments in body fluids. In contrast, the NEBNext small RNA library preparation kit aims to prepare small RNA libraries from “total RNA” instead of cfRNA for sequencing by Illumina sequencers. NEB's method requires at least 100 ng total RNA as the starting material to make a small RNA sequencing library. Furthermore, NEB's method uses two different adaptors and thus the downstream amplification requires two different primers which leads to lower efficiency. Illumina's method is not suitable for minute cfDNA sequencing and thus is not suitable for cfRNA/vcRNA sequencing either.

The advantages of the method of the present invention over NEB's approach include, but not limited to, the following: 1) the method of the present invention can assess cfRNAs including vcRNAs, although it is also applicable in RNAs in cells; 2) the method of the present invention needs less amount of RNAs as the initial input (about 1 ng or less is sufficient); 3) the method of the present invention can detect a large variety of RNA populations, not limited to certain types of RNAs; 4) the method of the invention can achieve a comprehensive RNA profile by converting a large variety of RNA species to the corresponding cDNAs in relative quantity in the sample, without bias; (5) the method of the present invention can provide increased sensitivity and less false negatives when applying in diagnosis; 6) the method of the present invention produces a single-type (homogeneous) adaptor, while NEB's method generates two (heterogeneous) adaptors; and 7) the method of the present invention amplifies RNA-derived cDNA by T-oligo-primed polymerase chain reaction (TOP-PCR) using single T oligo primer (which may use base U instead of base T in some positions). TOP-PCR is a superior and more efficient approach compared to Illumina's method (Nai et. al., 2017; Sci. Rep. 7: 40767).

The present invention is further illustrated by the following examples, which are provided for the purpose of demonstration rather than limitation. Those of skill in the art should, in light of the present disclosure, appreciate that many changes can be made in the specific embodiments which are disclosed and still obtain a like or similar result without departing from the spirit and scope of the invention.

### Examples

### 1. Materials and Methods

1.1 Cell-Free RNA Isolation

Cell-free RNA was isolated from the plasma of healthy males. Whole blood samples of a healthy male were collected in BD Vacutainer Venous Blood Collection tubes (BD, #367525). Plasma cfRNA fragments were isolated using miRNeasy Serum/Plasma Kit (Qiagen, #217184). Isolated cfRNA samples were quantified with Qubit RNA HS Assay kit (Thermo Fisher, #Q32852) and stored at −70° C. Fragment Analyzer (AATI) using either RNA or DNA gel was used to estimate quantify and quality of RNA and DNA samples.

1.2 Conversion of cfRNA to cDNA and Amplification to Obtain a dsDNA Product

FIG. 1 shows procedures of the process of the present invention including steps A to G.

The cfRNA samples were converted to cDNA by the following steps without purification.

Step A: 5′ Dephosphorylation of cfRNA

In step A, cfRNA was dephosphorylated at Send. 5 μL of dephosphorylation mixture contains 20 mM Tris-HCl (pH 8.0), 10 mM MgCl₂, 1 unit/μL of RNase Inhibitor (NEB, #M0314), and 1 unit of shrimp alkaline phosphatase (NEB, #M0371). The mixture was incubated for 30 min at 37° C. and 10 min at 65° C. As a result, cfRNA was dephosphorylated at the 5′end.

Step B: 3′ ligation of cfRNA to P oligo

In step B, P oligo was added and ligated to 3′end of dephosphorylated cfRNA. 18 μL of 3′ ligation mixture contains 50 mM Tris-HCl (pH 7.5), 10 mM MgCl₂, 1 mM DTT, 1 mM ATP, 11 nt P oligo (DNA) at 40× molar ratio (Sigma, 5′-phos-AGTCGGAGTCT (SEQ ID NO: 1)-[AmC3]-3′), 25% PEG 8000, 1 unit/μL of RNase Inhibitor, and 1 unit/μL T4 RNA ligase 1 (NEB, #M0437). The reaction mixture was incubated for 1 h at 37° C. and hold at 4° C. As a result, a cfRNA fragment ligated with P oligo at 3′end was obtained.

Step C: 1^{st} cDNA Synthesis by Reverse Transcription (RT)

In step C, T oligo (DNA form, complementary to P oligo) was added and annealed to the P oligo portion of the cfRNA fragment. The 30 μL of RT mixture contains 50 mM Tris-HCl (pH 8.3), 75 mM KCl, 6 mM MgCl₂, 10 mM DTT, 0.5 mM dNTP, 1 unit/μL of RNase Inhibitor and 100 units of ProtoScript II Reverse Transcriptase (NEB, #M0368)]. Prior to RT, 11 nt T oligo (DNA, complementary to P oligo) at 40× molar ratio (IDT, 5′-[AmMC6]-AGACTCCGACT (SEQ ID NO: 2)-3′) was added to 3′ end ligation mixture (from step B) and incubated for 5 min at 65° C., 5 min at 37° C., 5 min at 25° C., and hold at 4° C., leading to annealing T oligo to P oligo. Then, the reaction mixture was incubated for 10 min at 25° C., 50 min at 42° C., 20 min at 65° C. and hold at 4° C. As a result, a first strand cDNA was synthesized and a RNA/DNA hybrid including the first strand cDNA complementary to the cfRNA fragment with P oligo was formed.

Step D: 5′ Adaptor Ligation of cfRNA with T Oligo (RNA Form)

In step D, T oligo (RNA form) was added and ligated to 5′end of the cfRNA fragment in the RNA/DNA hybrid. 45 μL of phosphorylation mixture contains 50 mM Tris-HCl (pH 7.5), 10 mM MgCl₂, 10 mM DTT, 1.4 mM ATP, 20% PEG 8000, 1 unit/μL of RNase Inhibitor, and 10 units of T4 Polynucleotide Kinase (NEB, #M0201). The reaction mixture for phosphorylation was incubated for 30 min at 37° C. and hold at 4° C. Then, 11 nt T oligo in RNA form (IDT, 5′-AmMC6-rArGrArCrUrCrCrGrArCrU (SEQ ID NO: 3)-3′) was added to the phosphorylation mixture at 200× molar ratio and incubated for 5 min at 65° C., 5 min at 37° C., 5 min at 25° C., and hold at 4° C. Next, T oligo was ligated to 5′ end of cfRNA. A total of 60 μL ligation mixture contains 50 mM Tris-HCl (pH 7.5), 7.5 mM MgCl₂, 7.5 mM DTT, 1.8 mM ATP, 25% PEG 8000, 1 unit/μL of RNase Inhibitor, and 5 units of T4 RNA Ligase 2 (NEB, #M0239)]. The reaction mixture for ligation was incubated for 2 h at 37° C. and hold at 16° C.

Step E: Extended Reverse Transcription

In step E, extended reverse transcription was performed to form a complete RNA-DNA duplex. 75 μL of extended RT mixture contains 50 mM Tris-HCl (pH 8.3), 75 mM KCl, 6 mM MgCl₂, 10 mM DTT, 0.4 mM dNTP, 1 unit/μL of RNase Inhibitor and 100 units of ProtoScript II Reverse Transcriptase. The reaction mixture was incubated for 20 min at 42° C., 20 min at 65° C., and hold at 4° C. As a result, a complete RNA/DNA hybrid was formed.

Step F: RNA Digestion

In step F, RNase was added to digest the RNA fragment in the RNA/DNA hybrid. a total of 7.5 units RNase H (NEB, #M0297) and 7.5 μg RNase A (QIAGEN, #19101) was added to extended RT mixture (from step E), then incubated for 20 min at 37° C., 20 min at 65° C. and hold at 4° C. to remove RNA, leaving the DNA fragment only prior TOP-PCR amplification step.

Step G: TOP-PCR Amplification

In step G, the DNA fragment (without P oligo after denaturation) was used as a template and T-3U oligo (IDT, 5′-AGCGCUAGACUCCGACU-3′) (SEQ ID NO: 4) was used as a single primer to perform PCR amplification to obtain a dsDNA product.

750 μL of PCR mixture contains 1× Phusion HF buffer, 0.2 mM dNTP, 1 μM 17 nt T-3U oligo, and 15 units of Phusion U Hot Start DNA Polymerase (ThermoFisher, #F555)]. The PCR condition: 1) 1 cycle of initial denaturation at 98° C. for 30 sec; 2) 3-5 cycles of denaturation at 98° C. for 10 sec, primer annealing at 27° C. for 1 min, and extension at 72° C. for 1 min; 3) 15-20 cycles of denaturation at 98° C. for 10 sec, primer annealing at 57° C. for 30 sec, and extension at 72° C. for 1 min; and 4) Final extension at 72° C. for 5 min and hold at 4° C. PCR product was treated with Exonuclease I (NEB, #M0293) to remove primer and purified with QIAquick Nucleotide Removal Kit (QIAGEN, #28304). Adaptor-ligated dsDNA was quantified with Qubit™ DNA HS Assay kit (ThermoFisher, #Q32851) and stored at −70° C.

T-3U oligo is removed before sequencing library construction.

1.3 Sequencing Library Preparation and Sequencing

Adapters used in TOP-PCR had to be removed prior to sequencing library construction. To make a sequencing library, ˜10 ng of DNA generated from previous steps were treated with 2 units of Thermolabile USER II enzyme (NEB, M5508) in 25 μL of 1×TE buffer (10 mM Tris-HCl pH 8.0, 0.1 mM EDTA), then incubated at 37° C. for 15 min and hold at 25° C. to completely remove the adapters. Illumina sequencing libraries were constructed by using NEBNext Ultra II DNA Library Prep Kit (NEB, E7645) following manufacturer's instructions. The sequencing library was quantified with Qubit DNA HS Assay kit and stored at −20° C.

Sizes were estimated by Agilent Fragment Analyzer and quantification was measured by Roche LightCycler LC480 II machine using qPCR-based KAPA Library Quantification Kit (Roche, KK4854). Libraries were sequenced with 2×150 bp paired-end (PE) sequencing using HiSeq X Ten (Macrogen, South Korea).

1.4 Processing of Raw Reads

Potential carryover of adapter sequences formed by P and T-3U oligos were removed from raw reads by Cutadapt software. P5 and P7 adapters used for Illumina sequencing were also trimmed by Cutadapt. Software PRINSEQ was then used to examine base quality score and the presence of ambiguous base (N). Read quality was then examined by NGS QC Toolkit with default parameter. For each step, the minimal read length is 15. FLASH with defined parameters (-m 4 -M 151) was applied to combined paired reads into a fragments.

1.5 Mapping and Sequence Analysis

Quality reads were mapped to human genome GRCh38.p12 using RNA-seq aligner STAR (Dobin et al., 2013). In addition, GENCODE reference annotation (release 29) was employed to identify genes in the human genome (Frankish et al., 2019). Gene-associated reads were calculated and analyzed for further analysis by featureCounts software (Liao et al., 2014). Post-processing of SAM/BAM files was executed by SAMtools (Li et al., 2009), and statistics information was generated from BAM files using the Picard tools (https://broadinstitute.2ithub.io/picard).

### 2. Results

2.1 cfRNA Assessment

A cfRNA sample was isolated from the plasma of each of three healthy males and subjected to the RNA TOP-PCR method of the present invention. For read quality control, we applied QV value of 20 as the cutoff. Table 1 shows the results.

TABLE 1
Origins of cell-free RNAs

| | | Libraries | | |
|---|---|---|---|---|
| | | Male-1 | Male-2 | Male-3 |
| # Quality PE reads | | 114,751,125 | 149,594,241 | 178,304,241 |
| Origins | Mitochondria | 12,770,790 (12.4) | 6,884,109 (5.1%) | 1,098,624 (0.7%) |
| of | rRNA | 59,537,165 (57.6%) | 59,568,493 (43.7%) | 105,099,757 (64.9%) |
| cfRNAs | tRNA | 13,932 (0.01%) | 58,387 (0.04%) | 154,575 (0.10%) |
| | mRNA | 15,615,460 (15.1%) | 35,709,856 (26.2%) | 27,354,378 (16.9%) |
| | lncRNA | 6,079 (0.01%) | 10,033 (0.01%) | 2,985 (0.00%) |
| | YRNA | 47,991 (0.05%) | 41,678 (0.03%) | 10,017 (0.01%) |
| | Vault RNA | 280 (0.00%) | 27 (0.00%) | 33 (0.00%) |
| | Unmapped | 15,430,147 (14.9%) | 34,058,019 (25.0%) | 28,172,812 (17.4%) |

The major sources of cfRNA fragments are 1) rRNA, followed by 2) mRNA, 3) mitochondrial RNA, and 4) YRNA. Of particular interest is YRNA, which is known to involve in immunity.

It is demonstrated that the method of the present invention is capable of converting trace amounts of cfRNA fragments into DNA fragments that can be subjected to amplification and/or sequencing to generate a comprehensive RNA profile and facilitate biological study and analysis of RNA species, for example, for diagnosis and early detection of diseases.

2.2 EV-RNA Assessment

2.2.1 Workflow

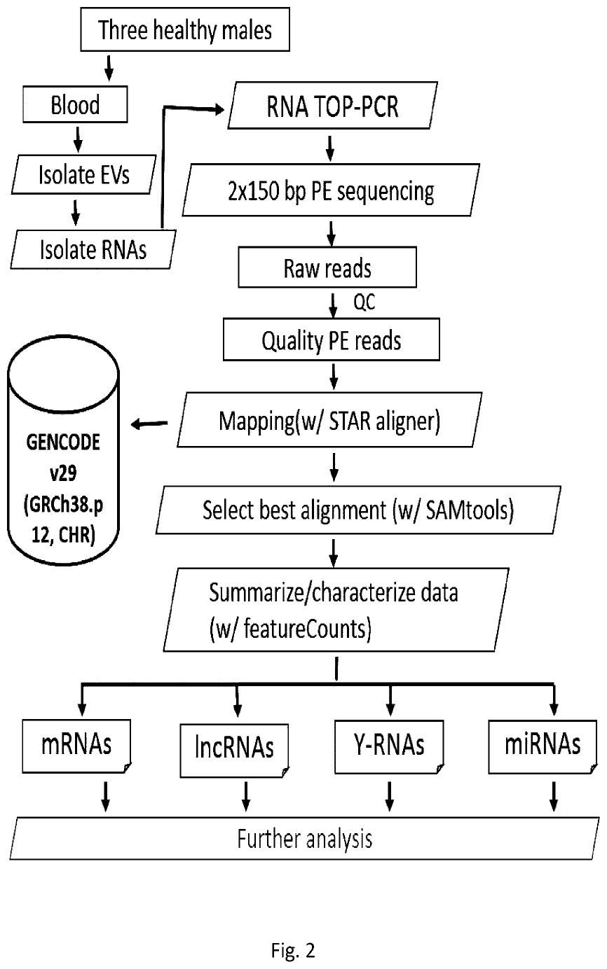

A workflow is outlined below to illustrate the process of extracellular vesicle RNAs (EV-RNAs) sequencing (FIG. 2). Briefly, EV-RNAs were isolated from EVs and subjected to RNA TOP-PCR, which converted RNAs to cDNAs, followed by TOP-PCR amplification. The process was performed in a single-tube to prevent loss of precious material. Adapters in amplified cDNAs were removed by enzymatic digestion and the cDNAs were sequenced by NGS. Quality reads were mapped to GENCODE database to identify sequence origins in human genome. Data were then categorized by featureCounts. Sequences of mRNAs, lncRNAs, Y-RNAs and miRNAs were further analyzed.

2.2.2 Library Statistics and Size Distribution

An EV-RNA samples was isolated from the whole blood of each of three healthy males and subjected to the RNA TOP-PCR method of the present invention. Library statistics are shown in Table 2. Only R1-R2 paired mappable reads were used in this study.

TABLE 2
Library statistics and molecular compositions of EV-RNA libraries

| | | Library IDs of the individuals studied | | |
|---|---|---|---|---|
| Description of reads | | M1 | M2 | M3 |
| Raw PE reads | | 146,305,550 | 157,173,135 | 160,462,457 |
| Quality PE | Total | 77,564,041 | 106,202,280 | 38,429,918 |
| reads | w/overlap | 72,042,366 | 95,664,333 | 35,015,882 |
| | w/o overlap | 5,521,675 | 10,537,947 | 3,414,036 |
| | Mappable | 76,264,355 | 105,021,697 | 26,958,493 |

All EV-RNAs were analyzed for size distribution. Profiling of the fragment sizes in EV-RNA samples revealed two major regions (data not shown). The major peak ranges between 150-170 bases, mainly formed by rRNAs and mRNAs, while the second region ranges between 90-110 bases, mainly constituted by Y-RNAs and tRNAs (72-80 bases, 87-89 bases (major) and 120-126 bases).

2.2.3 EV-RNAs Comprise Diverse RNA Species

As revealed by featureCounts, all EV-RNA collections contain broadly diverse RNA species (Table 3).

TABLE 3
Summary of annotated EV-RNAs

| Category | Origins | M1 | % | M2 | % | M3 | % |
|---|---|---|---|---|---|---|---|
| mRNA | protein coding | 575,271 | 0.8 | 730,658 | 0.7 | 459,939 | 1.7 |
| | IG_C_gene | 405 | 0.0 | 302 | 0.0 | 90 | 0.0 |
| | IG_D_gene | — | 0.0 | — | 0.0 | — | 0.0 |
| | IG_J_gene | — | 0.0 | — | 0.0 | — | 0.0 |
| | IG_V_gene | 173 | 0.0 | 104 | 0.0 | 252 | 0.0 |
| | TR_C_gene | 103 | 0.0 | 198 | 0.0 | 73 | 0.0 |
| | TR_D_gene | — | 0.0 | — | 0.0 | — | 0.0 |
| | TR_J_gene | — | 0.0 | 43 | 0.0 | 62 | 0.0 |
| | TR_V_gene | 18 | 0.0 | 62 | 0.0 | 178 | 0.0 |
| rRNA | rRNA | 23,721,471 | 31.1 | 45,737,594 | 43.6 | 10,739,589 | 39.8 |
| tRNA | tRNA | 113,933 | 0.1 | 69,583 | 0.1 | 27,809 | 0.1 |
| Mt_rRNA | Mt_rRNA | 4,684,838 | 6.1 | 4,212,871 | 4.0 | 437,174 | 1.6 |
| Mt_tRNA | Mt_tRNA | 24,638 | 0.0 | 10,163 | 0.0 | 2,297 | 0.0 |
| miRNA | miRNA | 51 | 0.0 | 162 | 0.0 | 1,030 | 0.0 |
| Y-RNA | Y-RNA | 7,119,141 | 9.3 | 4,068,465 | 3.9 | 1,685,919 | 6.3 |
| Long non- | processed_transcript | 627 | 0.0 | 1,256 | 0.0 | 5,518 | 0.0 |
| coding RNA | lincRNA | 4,619,995 | 6.1 | 8,275,637 | 7.9 | 1,104,098 | 4.1 |
| | 3prime_overlapping_ncRNA | 41 | 0.0 | — | 0.0 | 77 | 0.0 |
| | antisense | 1,377 | 0.0 | 2,520 | 0.0 | 25,508 | 0.1 |
| | non_coding | — | 0.0 | — | 0.0 | 14 | 0.0 |
| | sense_intronic | 146 | 0.0 | 355 | 0.0 | 2,929 | 0.0 |
| | sense_overlapping | 194 | 0.0 | 530 | 0.0 | 1,222 | 0.0 |
| | TEC | 101 | 0.0 | 442 | 0.0 | 5,236 | 0.0 |
| | known_ncrna | — | 0.0 | — | 0.0 | — | 0.0 |
| | macro_lncRNA | — | 0.0 | 1 | 0.0 | 301 | 0.0 |
| | bidirectional_promoter_lncRNA | 178 | 0.0 | 619 | 0.0 | 1,027 | 0.0 |
| | lncRNA | — | 0.0 | — | 0.0 | — | 0.0 |
| Small non- | snRNA | 25,589 | 0.0 | 32,939 | 0.0 | 8,961 | 0.0 |
| coding RNA | snoRNA | 5,422 | 0.0 | 6,356 | 0.0 | 2,761 | 0.0 |
| | misc_RNA | 855,309 | 1.1 | 721,280 | 0.7 | 84,783 | 0.3 |
| | ribozyme | — | 0.0 | — | 0.0 | — | 0.0 |
| | sRNA | — | 0.0 | — | 0.0 | — | 0.0 |
| | scRNA | 6 | 0.0 | 103 | 0.0 | 1 | 0.0 |
| | scaRNA | 905 | 0.0 | 1,002 | 0.0 | 500 | 0.0 |
| | vaultRNA | — | 0.0 | — | 0.0 | — | 0.0 |
| Pseudogenes | processed_pseudogene | 6,736 | 0.0 | 6,559 | 0.0 | 28,022 | 0.1 |
| | transcribed_processed_pseudogene | 290 | 0.0 | 717 | 0.0 | 3,281 | 0.0 |
| | translated_processed_pseudogene | 4 | 0.0 | 1 | 0.0 | 1 | 0.0 |
| | unprocessed_pseudogene | 13,729 | 0.0 | 13,229 | 0.0 | 12,435 | 0.0 |
| | transcribed_unprocessed_pseudogene | 1,651 | 0.0 | 2,310 | 0.0 | 10,106 | 0.0 |
| | unitary_pseudogene | — | 0.0 | 13 | 0.0 | 356 | 0.0 |
| | transcribed_unitary_pseudogene | 166 | 0.0 | 199 | 0.0 | 1,669 | 0.0 |
| | polymorphic_pseudogene | 553 | 0.0 | 602 | 0.0 | 513 | 0.0 |
| | pseudogene | — | 0.0 | — | 0.0 | — | 0.0 |
| | rRNA_pseudogene | 9,286 | 0.0 | 12,412 | 0.0 | 2,399 | 0.0 |
| | IG_C_pseudogene | — | 0.0 | — | 0.0 | 1 | 0.0 |
| | IG_J_pseudogene | — | 0.0 | — | 0.0 | — | 0.0 |
| | IG_pseudogene | — | 0.0 | — | 0.0 | — | 0.0 |
| | IG_V_pseudogene | 7 | 0.0 | — | 0.0 | 437 | 0.0 |
| | TR_J_pseudogene | — | 0.0 | — | 0.0 | — | 0.0 |
| | TR_V_pseudogene | — | 0.0 | — | 0.0 | 24 | 0.0 |
| | tRNA_pseudogene | — | 0.0 | — | 0.0 | 73 | 0.0 |
| | | 41,782,354 | 54.8 | 63,909,287 | 60.9 | 14,656,665 | 54.4 |

%, percentage over number of mappable reads.

We further associated the EV-RNA species into a few major groups (Table 4). In general, rRNA constitutes the major group followed by Y-RNA. Contrarily, miRNA constitutes the smallest group, likely due to loss in initial RNA isolation from EVs (the kit we used was not aiming for miRNA analysis).

TABLE 4
Major groups of EV-RNAs

| | | Group | | | | | |
|---|---|---|---|---|---|---|---|
| | | M1 | | M2 | | M3 | |
| | Total mappable reads | 76,264,355 | % | 105,021,697 | % | 26,958,493 | % |
| 1 | rRNA | 23,721,471 | 31.1 | 45,737,594 | 43.6 | 10,739,589 | 39.8 |
| 2 | tRNA | 113,933 | 0.1 | 69,583 | 0.1 | 27,809 | 0.1 |
| 3 | Mt_rRNA | 4,684,838 | 6.1 | 4,212,871 | 4.0 | 437,174 | 1.6 |
| 4 | Mt_tRNA | 24,638 | — | 10,163 | — | 2,297 | — |
| 5 | mRNA | 575,970 | 0.8 | 731,367 | 0.7 | 460,594 | 1.7 |
| 6 | Long non-coding RNA | 4,622,659 | 6.1 | 8,281,360 | 7.9 | 1,145,930 | 4.3 |
| 7 | Small non-coding RNA | 887,231 | 1.2 | 761,680 | 0.7 | 97,006 | 0.4 |
| 8 | Y-RNA | 7,119,141 | 9.3 | 4,068,465 | 3.9 | 1,685,919 | 6.3 |
| 9 | miRNA | 51 | — | 162 | — | 1,030 | — |
| 10 | Pseudogenes | 32,422 | — | 36,042 | — | 59,317 | 0.2 |
| | Annotated total (sum of | 41,782,354 | 54.7 | 63,909,287 | 60.9 | 14,656,665 | 54.4 |
| | above) | | | | | | |
| | Unannotated total | 34,482,001 | 45.3 | 41,112,410 | 39.1 | 12,301,828 | 45.6 |

%, percentage over number of mappable reads; mRNA (protein-coding genes with mitochondrial genes included, Igb, TCR).

2.2.4 EV-mRNAs Derive from Thousands of Protein-Coding Genes

The PV-mRNAs in these three healthy males tested were transcribed from a total of ˜15,000 protein-coding genes where these three individuals shared (0.25% overlap between these genes (0 refers to percentage over total number of 14,851 genes, data not shown).

We further conducted pathway analysis using IPA together with EV-mRNAs associated with protein-coding genes shared by all three individuals (3,688 total). The results show that the top 5 pathways are all associated with signal transduction (Table 5).

TABLE 5
Pathway analysis based on 3688 genes shared by all three.
EV-mRNAs

| Pathway | p-value | Overlap |
|---|---|---|
| EIF2 Signaling | 5.17E−65 | 149/224 (66.5%) |
| Regulation of eIF4 and | 1.82E−40 | 99/157 (63.1%) |
| p70S6K Signaling | | |
| mTOR Signaling | 7.79E−36 | 112/210 (53.3%) |
| Integrin Signaling | 7.73E−30 | 105/213 (49.3%) |
| Estrogen Receptor Signaling | 2.09E−28 | 136/328 (41.5%) |

Another independent pathway study using IPA together with the top-5000 genes from each, weighed by number of associated reads, also showed similar result (Table 6).

TABLE 6
IPA on each individual.

| | | |
|---|---|---|
| Pathway | p-value | Overlap |

M1

| | | |
|---|---|---|
| EIF2 Signaling | 1.05E−53 | 156/224 (69.6%) |
| Regulation of eIF4 and | 7.31E−36 | 107/157 (68.2%) |
| p70S6K Signaling | | |
| mTOR Signaling | 5.03E−32 | 124/210 (59.0%) |
| Integrin Signaling | 9.38E−26 | 116/213 (54.5%) |
| ERK/MAPK Signaling | 2.26E−25 | 108/193 (56.0%) |

M2

| | | |
|---|---|---|
| EIF2 Signaling | 1.45E−54 | 157/224 (70.1%) |
| Regulation of eIF4 and | 3.51E−33 | 104/157 (66.2%) |
| p70S6K Signaling | | |
| Integrin Signaling | 5.16E−27 | 118/213 (55.4%) |
| Molecular Mechanisms | 1.03E−25 | 177/391 (45.3%) |
| of Cancer | | |
| Estrogen Receptor | 4.57E−25 | 155/328 (47.3%) |
| Signaling | | |

M3

| | | |
|---|---|---|
| EIF2 Signaling | 1.86E−19 | 109/224 (48.7%) |
| Molecular Mechanisms | 1.90E−14 | 150/391 (38.4%) |
| of Cancer | | |
| Protein Kinase | 1.97E−14 | 152/398 (38.2%) |
| A Signaling | | |
| GNRH Signaling | 1.15E−13 | 81/173 (46.8%) |
| Integrin Signaling | 9.61E−13 | 92/213 (43.2%) |

To evaluate data reliability through reproducibility, we identified and compared the top 50 protein-coding genes in three individuals. We found that, in any individual, over 50% of top 50 protein-coding genes are also shared by other individuals, suggesting a high degree of reproducibility among these individuals (data not shown). High prevalence of mitochondrial originated sequences also indicated a selectivity of particular mitochondrial sequences, especially those encoding NADH dehydrogenase isoforms.

2.2.5 Y-RNA/RNY Analysis

There are four Y RNAs inhuman. These Y RNAs are known to be a repressor of Ro 60-kDa, a helical HEAT repeat-containing RNA-binding protein, and initiation factor of DNA replication, and biogenesis of small RNA from Y RNA is independent of miRNA (Nicolas et al., 2012). Each type Y RNA contains loop domain, upper stem domain, lower stem domain, and polyuridine tail.

Our results showed that RNY3 and RNY4 are the major Y-RNA species in EVs, followed by RNY1, while RNY5 is very minor (Table 7).

TABLE 7
Y-RNA species of all subjects

| Gene name | M1 | M2 | M3 |
|---|---|---|---|
| RNY1 | 913,042 | 593,943 | 179,880 |
| RNY3 | 3,301,852 | 1,573,633 | 643,852 |
| RNY4 | 2,902,204 | 1,899,113 | 861,435 |
| RNY5 | 2,043 | 1,776 | 752 |
| | 7,119,141 | 4,068,465 | 1,685,919 |

2.2.6 Comparison of Our Data with Previously Reported Data

We compared our results with previous reports (Table 8). Most of the reports, which were also produced from blood plasma-harbored EVs of healthy persons, focused on small or long RNAs in EVs (Ferrero et al., 2018; Li et al., 2019; Yuan et al., 2016). Here, we compare our results with the report by Everaert et al (Everaert et al., 2019), focusing on the analysis of total EV-RNAs.

TABLE 8
Comparison of plasma-derived EV-RNA profile from heathy individuals.

| | | | EV/isolation method | |
|---|---|---|---|---|
| | | | RNA extraction method | |
| | | | Library prep. (read length) | Results |
| | | | Annotation database | (only annotated |
| Reference | Purpose | subject | (alignment tool) | RNAs are included) |
| 1. Our | Total EV- | 3 | exoRNeasy Serum/ | mRNA 7.59% |
| current | RNA | | Plasma kit (Qiagen) | IncRNA 42.27% |
| study | profiling | | RNA TOP-PCR + | small non-coding |
| | | | NEBNext Ultra II | RNA 4.96% |
| | | | DNA Library Prep Kit | YRNA 43.61% |
| | | | (Illumina) (2 × 150 bp) | pseudogenes 0.74% |
| | | | GENCODE v29 (STAR) | miRNA 0.01% |
| | | | | tRNA 0.83% |
| 2. | Total RNA | 1 | Size exclusion chromatography + | mRNA 70% |
| Everaert | profiling | | OptiPrep density gradient | IncRNA 10.8% |
| et al., | | | centrifugation | miscRNA 14% |
| 2019 | | | miRNeasy Serum/ | pseudogenes 3.3% |
| | | | Plasma Kit (Qiagen) | other 1.6% |
| | | | Stranded SMARTer | |
| | | | total RNA-seq | |
| | | | kit (Clontech) (2 × 75 bp) | |
| | | | GENCODE (STAR) | |

There were significant differences between our experimental procedure and that employed by Everaert et al. First of all, they pre-excluded rRNA during library preparation step, while, aiming to compare the EV-RNA profile with them, we masked rRNA here. Secondly, they performed fragmentation on RNAs prior to cDNA synthesis, while we directly used the original EV-RNAs in a single-tube procedure, where no fragmentation nor purification was involved until TOP-PCR amplification was finished. Such variations in experimental procedure may be the major reasons causing the differences in outcome.

### 3. Discussion

It is well-known that cfRNAs present in biological fluids are valuable genetic material for the diagnosis of many diseases including cancer. However, cfRNAs are usually fragmented, of low abundance and of diverse varieties, making the identification and assessment of cfRNAs a great challenge. Most previous reports focused on certain types of RNAs associated with particular diseases, while there are numerous cfRNAs potentially involved in different physiological processes and/or diseases but not yet identified or studied.

In this study, we have developed a new RNA TOP-PCR method for comprehensive analysis of RNAs from biological samples of individuals. As a method designed for amplification of minute quantity of RNAs, the RNA TOP-PCR method of the present invention possesses a number of advantages, including the single-tube procedure, which prevents loss of sample by eliminating RNA/cDNA isolation until amplification is complete. Moreover, adapters can be removed after amplification so that the sample can be directly subjected to sequencing or be used for diagnosis with conventional methods. The RNA TOP-PCR method of the present invention.

We have demonstrated that the RNA TOP-PCR method of the present invention is workable for comprehensive amplification and detection of total cfRNAs from biofluid samples of individuals.

Blood vessels in cardiovascular circulation act like a super canal system allowing the body to achieve a bodywise homeostasis potentially for all physiological aspects. In the blood circulatory system, similar to red blood cells that carry oxygen molecules, EVs act like molecular cargos for systematic transport of particular molecules between cells. In the process, nucleic acids such as EV-mRNAs and EV-ncRNAs are known to retain their coding and regulatory activities, respectively for intercellular coordination in gene expression and regulation. Studies of EV-RNAs have gradually unraveled a horizontal coordination in gene expression per se as well as the regulation of gene expression, extending from intracellular to intercellular level.

It is important to have an independent approach or method for EV-RNAs analysis. With the present RNA TOP-PCR, we identified not only the previously reported ncRNAs but also large amount of novel ncRNA transcription sites in human genome. Most previous studies focused on one or a few species of EV-RNAs, while here, taking advantage of the unbiased nature of RNA TOP-PCR, we intended to survey all RNA species in EVs. To avoid overestimation of RNA level, no fragmentation was involved in sample preparation.

Notice that, quality of EV-RNA sequencing is first influenced by methods used for EV and RNA isolation and later by methods used for sequencing library preparation. Certain “selective” reagent kits allow researchers to focus on specific RNA species such as miRNA or mRNA and at the same time ignore the rest. Moreover, the downstream sequence data analysis are also influenced by the mapping tool, databases used and bioinformatics approaches.

We identified a large amount of EV-mRNAs and found that these mRNA sequences belong to about 15,000 protein-coding genes which also mainly involved in signal transduction. There was a high degree of overlap among top 50 EV-mRNA-coding genes between these males (44% shared by all three plus 8-40% shared by any two of them). Furthermore, most of the top 20 EV-mRNAs encode subunits of NADH dehydrogenase, which are normally destined to the inner membrane of mitochondria.

### REFERENCES

Dobin, A., Davis, C. A., Schlesinger, F., Drenkow, J., Zaleski, C., Jha, S., Batut, P., Chaisson, M., and Gingeras, T. R. (2013). STAR: ultrafast universal RNA-seq aligner. Bioinformatics 29, 15-21.
Everaert, C., Helsmoortel, H., Decock, A., Hulstaert, E., Van Paemel, R., Verniers, K., Nuytens, J., Anckaert, J., Nijs, N., Tulkens, J., et al. (2019). Performance assessment of total RNA sequencing of human biofluids and extracellular vesicles. Sci Rep 9, 17574.
Ferrero, G., Cordero, F., Tarallo, S., Arigoni, M., Riccardo, F., Gallo, G., Ronco, G., Allasia, M., Kulkami, N., Matullo, G., et al. (2018). Small non-coding RNA profiling in human biofluids and surrogate tissues from healthy individuals: description of the diverse and most represented species. Oncotarget 9, 3097-3111.
Frankish, A., Diekhans, M., Ferreira, A. M., Johnson, R., Jungreis, I., Loveland, J., Mudge, J. M., Sisu, C., Wright, J., Armstrong, J., et al. (2019). GENCODE reference annotation for the human and mouse genomes. Nucleic Acids Res 47, D766-D773.
Li, H., Handsaker, B., Wysoker, A., Fennell, T., Ruan, J., Homer, N., Marth, G., Abecasis, G., Durbin, R., and Genome Project Data Processing, S. (2009). The Sequence Alignment/Map format and SAMtools. Bioinformatics 25, 2078-2079.
Li, Y., Zhao, J., Yu, S., Wang, Z., He, X., Su, Y., Guo, T., Sheng, H., Chen, J., Zheng, Q., et al. (2019). Extracellular Vesicles Long RNA Sequencing Reveals Abundant mRNA, circRNA, and lncRNA in Human Blood as Potential Biomarkers for Cancer Diagnosis. Clin Chem 65, 798-808.
Liao, Y., Smyth, G. K., and Shi, W. (2014). featureCounts: an efficient general purpose program for assigning sequence reads to genomic features. Bioinformatics 30, 923-930.
Nicolas, F. E., Hall, A. E., Csorba, T., Tumbull, C., and Dalmay, T. (2012). Biogenesis of Y RNA-derived small RNAs is independent of the microRNA pathway. FEBS Lett 586, 1226-1230.
Yuan, T., Huang, X., Woodcock, M., Du, M., Dittmar, R., Wang, Y., Tsai, S., Kohli, M., Boardman, L., Patel, T., et al. (2016). Plasma extracellular RNA profiles in healthy and cancer patients. Sci Rep 6, 19413.

1. A method of converting a linear, single-stranded RNA (ssRNA) fragment to a DNA fragment and amplifying the DNA fragment, comprising
(a) removing 5′ phosphate from the ssRNA fragment to produce a de-phosphorylated ssRNA fragment;
(b) ligating a P oligo (DNA), a single-stranded DNA having a P oligo sequence and carrying a 5′-phosphate, to 3-end of the de-phosphorylated ssRNA fragment to form a ssRNA-P oligo (DNA) strand;
(c) performing a first reverse transcription by using the ssRNA-P oligo (DNA) strand as a template and adding a T oligo (DNA), a single-stranded DNA having a T oligo sequence that is complementary to the P oligo (DNA), as a primer, to synthesize a complementary DNA (cDNA) strand that is complementary to the ssRNA fragment to produce a cDNA-T oligo (DNA) strand and thus form an initial RNA/DNA hybrid composed of said ssRNA-P oligo (DNA) strand and the cDNA-T oligo (DNA) strand;
(d) ligating a T oligo (RNA), a single-stranded RNA complementary to the P oligo (DNA), to 5′-end of the ssRNA-P oligo (DNA) strand in the initial RNA/DNA hybrid, to form a T oligo (RNA)-ssRNA-P oligo (DNA) strand and thus form an intermediate RNA/DNA hybrid composed of said T oligo (RNA)-ssRNA-P oligo (DNA) strand and the cDNA-T oligo (DNA) strand, having a non-complementary T oligo (RNA) overhang;
(e) performing a second reverse transcription using the non-complementary T oligo (RNA) overhang as an extended template to obtain a complete cDNA strand having the T oligo sequence at 5′-end and the P oligo sequence at 3-end and thus form a complete RNA/DNA hybrid of said T oligo (RNA)-ssRNA-P oligo (DNA) strand and said complete cDNA strand;
(f) removing the ssRNA fragment and the T oligo (RNA) from the complete RNA/DNA hybrid to produce a partial, double-stranded DNA comprising said complete cDNA strand partially hybridized at its 5′-end with the P oligo (DNA); and
(g) performing a polymerase chain reaction (PCR) using such complete cDNA strand as a PCR template and a T oligo primer having the T oligo sequence to prime synthesis of a double-stranded DNA product.

2. The method of claim 1, wherein the ssRNA fragment comprises a nucleic acid sequence indicative of a healthy or diseased state of a subject.

3. The method of claim 1, wherein the ssRNA fragment is present in a sample from a subject.

4. The method of claim 3, wherein the sample is obtained from a body fluid.

5. The method of claim 3, wherein the sample is blood, urine, saliva, tears, sweat, breast milk, nasal secretions, amniotic fluid, semen, or vaginal fluid of the subject.

6. The method of claim 1, wherein the ssRNA fragment is cell-free RNAs (cfRNAs) or RNAs in vesicles (vc-RNAs).

7. The method of claim 1, wherein prior to step (d) the ssRNA-P oligo (DNA) strand is phosphorylated.

8. The method of claim 1, wherein in step (g), the T oligo primer is the only primer used in amplification.

9. The method of claim 1, wherein the ssRNA fragment is present as an initial input (total RNA) in an amount in a range of 0.01 ng to 100 ng or less.

10. The method of claim 9, wherein the ssRNA fragment is present as an initial input (total RNA) in an amount in a range of 0.01 ng to 10 ng or less.

11. The method of claim 1, wherein the ssRNA fragment is present as an initial input (total RNA) in an amount in a range of 0.01 ng to 100 ng or more.

12. The method of claim 1, further comprising detecting the amplified cDNA product.

13. The method of claim 12, wherein the detecting is performed by mass spectrometry, hybridization or sequencing.

14. The method of claim 1, which does not include a purification step.

15. A method for RNA assessment, comprising
(i) providing a biofluid sample from a subject, wherein the biofluid includes ssRNA fragments;
(ii) performing a method of claim 1 to convert the ssRNA fragments to DNA fragments and amplify the DNA fragments; and
(iii) analyzing the amplified DNA fragments for measurement of one or more characteristics of the amplified DNA fragments.

16. The method of claim 15, wherein the analyzing step includes sequencing, mapping and/or alignment.

17. A kit for performing the method of claim 1, comprising
(i) a de-phosphorylation reagent comprising an alkaline phosphatase and a de-phosphorylation buffer;
(ii) a ligation reagent comprising a ligase, a ligation buffer, the P oligo (DNA) and the T oligo (RNA);
(iii) a phosphorylation reagent comprising a kinase and a kinase buffer;
(iv) a reverse transcription reagent comprising a reverse transcriptase (RT), an RT buffer, dNTP and the T oligo (DNA);
(iv) a RNA digestion reagent comprising an RNase and an RNase buffer; and
(v) a PCR reagent comprising a DNA polymerase, a PCR buffer, dNTP, and the T oligo primer.
